(19) United States
(12) Patent Application Publication
(10) Pub. No.: US 2006/0135489 A1
(43) Pub. Date: Jun. 22, 2006

(54) Chemical compounds containing tocopherol and at least one additional pharmaceutical active substrate

(76) Inventors: Barbara Matuszczak, Innsbruck (AT); Manfred Windisch, Graz (AT)

Correspondence Address: YOUNG & THOMPSON, 745 SOUTH 23RD STREET, 2ND FLOOR, ARLINGTON, VA, 22202, US

(21) Appl. No.: 10/524,147

(22) PCT Filed: Jul. 1, 2004
(86) PCT No.: PCT/AT04/00234

§ 371 Date: Oct. 24, 2005

(30) Foreign Application Priority Data
Jul. 17, 2003 (AT) A 1127/2003

(51) Int. Cl. A61K 31/60; A61K 31/355; C07D 311/04

(52) U.S. Cl. 514/159; 514/458; 549/410

(57) ABSTRACT

Compounds in the form of their racemates, enantiomers or diastereomers with general formula I 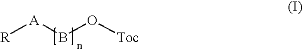
in which R stands for the unchanged portion of a variable pharmaceutical active ingredient molecule, B stands for a spacer, and Toc with the formula 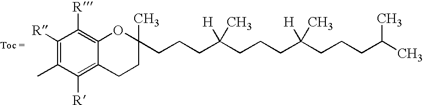
and R′, R″ and R′″ are equal to H or methyl stands for tocopherol and A stands for C═X, SOₘ, X or CH₂, whereby X is equal to 0, S or NR¹ (when n≧1) or S or NR¹ (when n=0), and B means a grouping X—R²—Y with Y equal to C═X, SOₘ or C(XR³)R⁴, and n is equal to 0 to 6, and m stands for 1 or 2, whereby R¹ stands for H, C₁ to C₁₀-alkyl, Het or an aryl or Het radical that is bonded via a C₁ to C₆-spacer, and whereby R² is selected from the group alkylene, arylene or Het spacers and combinations thereof, whereby the latter are linked to one another either directly or via radical A or via grouping Xₒ-A-Xₚ, whereby o and p are equal to 0, 1 or 2, and the latter can be the same or different, and whereby R³ and R⁴ stand for H, C₁ to C₁₀-alkyl, Het or an aryl or Het radical that is bonded via a C₁ to C₆ spacer.

The invention relates to chemical compounds that contain tocopherol as well as at least one other pharmaceutical active ingredient, process for the production of these chemical compounds as well as their use as pharmaceutical agents or prodrugs.

When using these chemical compounds as pharmaceutical agents or prodrugs, tocopherol exerts the action of an antioxidant; conversely, the other pharmaceutical active ingredient is preferably a non-steroidal anti-inflammatory agent (NSAID), which is linked directly to tocopherol or via a spacer. This “chemically-attached combination of two pharmaceutical active ingredients” results in more effective as well as more compatible derivatives. In the organism of the patient, the pharmaceutical active ingredient and tocopherol are released from the compounds claimed here by metabolic processes, such as the enzymatically catalyzed ester hydrolysis, and said active ingredient and tocopherol can then exert their known actions. The increase in effectiveness is produced from the optimization of the physicochemical parameters and the improved resorption produced therefrom and the uptake of the active ingredients by the central nervous system (CNS). The improved compatibility is primarily to be attributed to the reduction of possible, locally toxic effects, such as, for example, the reduction of locally-induced toxic effects of the NSAID components in the gastrointestinal tract by masking the carboxylic acid function, as well as the reduction of the active ingredient concentration in the periphery by increased uptake of compounds in the CNS.

In addition, the invention relates to a process for the production of the above-mentioned chemical compounds as well as their use as pharmaceutical substances or prodrugs for treatment or prophylaxis of degenerative diseases of the central nervous system, such as Alzheimer's disease, Lewy Body dementia, Parkinson's disease, Huntington's disease (chorea), multisystem atrophy and other similar diseases, such as also in the case of diseases caused by TNF (tumor necrosis factor)-alpha, IL (interleukin)-1 beta, IL (interleukin)-6 and/or IL (interleukin)-8 or other infirmities such as pain, diabetes, etc. Also and in particular, the use of the chemical compounds according to the invention in the production of pharmaceutical agents for the treatment of diseases that are influenced by radical stress, such as in diseases of the respiratory system, such as lung inflammation, of the digestive system, of the vascular system, such as leukemia, hemoglobinopathy, of the connective tissue, such as rheumatism, of the eyes, such as in cataracts, are subjects of the invention. The chemical compounds according to the invention are suitable expressly for the production of pharmaceutical agents for the treatment and prophylaxis of diseases in which inflammations and/or oxidative stress occur. The invention therefore comprises the production and the use of these chemical compounds in the case of all conditions covered here by introductory clauses and mentioned below.

Below, the medical background of the invention is explained in more detail.

In several respects, inflammatory processes play an essential role in the above-mentioned neurodegenerative diseases. In earlier works, it was postulated that inflammatory processes only occur in the brain in the case of damage to the blood-brain barriers. Later, however, it was proven that the brain can be put into operation and maintain inherent inflammatory processes.

It is now known that inflammation processes are involved very decisively at the beginning of the disease and as the disease progresses especially in the case of Alzheimer's disease. This is confirmed by a number of epidemiological studies (McGeer, 1992, Akiyama 2000). The thesis that NSAIDs have a positive effect on the course of Alzheimer's disease is also supported in that in the cortex of Alzheimer patients and older control patients, who had both neurofibrillar tangles (NFTs) and β-amyloid plaque, the estimated number of synapses, determined based on immunohistochemical data or loss of synapses, correlates much more strongly with inflammation markers than with the presence of NFTs and B-amyloid deposits (Rogers et al. 1995).

Even in the case of Alzheimer's disease, inflammation reactions are sometimes a sequela of the damage that sometimes already exists. Nevertheless, the brain in the case of Alzheimer's disease, as in several inflammatory diseases, such as asthma, arthritis, . . . in other body regions offers a number of possibilities for inflammations to develop whereby said inflammations can then cause greater damage than the original pathological changes. In many cases, it is assumed that β-amyloid plaques, however, are not necessarily sufficient for triggering and for advancing Alzheimer's disease. In this connection, inflammation reactions are a highly probable complementary factor that is also necessary for the clinical picture to develop (Rogers et al. 1995). It is advantageous that the toxicity of β-amyloid after the activation of complement proteins occurring in the brine increases by up to 1000× (Shalit et al. 1994). Aggregated β-amyloid is significantly more toxic than—more readily soluble—non-aggregated β-amyloid. It was possible to demonstrate in vitro that the complement protein Clq enhances the aggregation of p-amyloid (Webster et al. 1994). This seems especially important if it is considered that aggregated β-amyloid activates Clq (Jiang et al. 1994). Also, tau pathology, which plays an essential role in neurodegeneration in addition to β-amyloid, is closely associated with inflammatory processes and the activation of the complement system (Shen et al. 2001).

In the case of inflammatory processes, pro-inflammatory cytokines, such as interleukin 1, tumor-necrosis-factor alpha of various cell types, are released as a response to corresponding stimuli (in which, for example, lipopolysaccharide as well as various forms of cell stress are included). In addition to the above-mentioned neurodegenerative processes, an elevated release of the above-mentioned cytokines is associated with various diseases, such as, for example, rheumatoid arthritis, Paget's disease, osteoporosis, multiple myeloma, uveitis, acute or chronic myelogenic leukemia, loss of Beta cells, also as accompanying manifestations of insulin-dependent Type I diabetes, osteoarthritis, rheumatoid spondylitis, uratic arthritis, inflammatory intestinal diseases, respiratory distress syndrome of adults, psoriasis, Crohn's disease, allergic rhinitis, ulcerative colitis, anaphylaxis, contact dermatitis, asthma, muscle degeneration, cachexia, Reiter's syndrome, Type I and Type II diabetes, rejection reactions, reperfusion damage after ischemia, arteriosclerosis, cerebral trauma, multiple sclerosis, cerebral malaria, sepsis, septic shock, toxic shock syndrome, infection-induced fever and myalgia as well as infections with various viruses (HIV 1, HIV 2, HIV 3, CMV, influenza viruses, adeno viruses and herpes viruses. The invention therefore also relates to the use of chemical compounds according to the invention for the production of pharmaceutical agents for treating the above-mentioned diseases.

In neurodegenerative diseases, oxidative stress represents an especially important factor both in the initial stage and later (Butterfield et al. 2002). A number of anatomical, physiological and biochemical properties suggest that especially the central nervous system is at risk with respect to the damage caused by radicals: the brain consumes an especially large amount of oxygen in comparison to the other body regions. Expressed in numbers, this means a proportion of 20% of the total O₂ requirement at only 2% as a proportion of body weight. The result is an especially large potential for radicals to develop. In this connection, it was demonstrated that several cellular components are altered by oxidative stress: Proteins (Markesbery and Carney 1999), lipids (Sayre et al. 1997, Montine et al. 1998, McKracken et al. 2001), nuclear as well as mitochondrial DNA (Mecocci et al. 1994, Gabbita et al. 1998) and RNA (Nunomura et al. 1999) are—as repeatedly confirmed by literature—affected. Regarding preventive measures, the reduction of oxidative stress to reduce the risk of stroke is very useful (Chen and Zhou 2001, Mattson et al. 2001). Also, however, radical oxygen compounds (ROS) are produced during and directly after ischemia and have a harmful effect on the survival of nerve cells. The cell-biological changes that result therefrom in most cases last longer that the excitoxicity itself. In the course of lipid peroxidation that occurs in the hypoxia, toxic reaction products are produced, such as aldehyde 4-hydroxynonenal (McKracken et al. 2001), which creates both necrotic and apoptotic cell death. Also, it is highly probable that other factors that occur in the acute phase can be positively influenced to a decisive extent by substances that have an antioxidative action (El Kossi and Zakhary, M. M., 2001). Oxidative stress thus plays a significant role in the case of damage caused by stroke both in the first hours and even days later with long-lived reaction products.

By measurements of the 8-hydroxyguanosine (8OHG) content, it was possible to confirm that elevated oxidative stress is a very early feature of Alzheimer's disease (Nunomura et al. 2001). The development of the main components of the two most recognized theories for Alzheimer's disease, both the β-amyloid pathology and the tau-pathology, are, as confirmed by several bibliographic references, obviously narrow in connection with oxidative stress (Pappolla, M. A. et al. 2002). Especially in the early phases of Alzheimer's disease—even before the development of extracellular β-amyloid deposits—it results in the intracellular concentration of β-amyloid (Gouras et al. 2000). Since the oxidative stress is also especially pronounced during this early stage of the disease, a connection, for example, via metal ions that are bonded to β-amyloid (Nunomura et al. 2001) or neurofibrillar tangles (Sayre et al. 2000) and that can then form hydrogen peroxide directly, is very probable. The malfunction of mitochondria is another explanation that can be readily documented for the radical stress that occurs so early in an enhanced form (Hirai et al. 2001).

α-Synuclein, the protein that is also strongly prone to aggregation, which is the focal point of the pathology of Parkinson's disease, also results in the intensification of oxidative stress. In vivo studies and in vitro studies, even sometimes not directly associated with α-synuclein, confirm that oxidative stress is an early and very marked, detectable parameter in the development of Parkinson's disease (Migliore et al. 2002, Munch et al., 2000, Roghani and Behzadi 2001).

In addition to the above-mentioned diseases in the area of neurodegeneration, oxidative stress can result in arrythmias, myocardial infarction, arteriosclerosis, inflammation of the lungs, cerebral edemas, hemorrhagic and non-hemorrhagic infarctions, such as stroke, diseases of the gastric mucous membrane, the pancreas, cirrhoses, leukemia, hemoglobinopathy, sepsis, various forms of diabetes, stress reactions, diseases of the excretion system, such as inflammation of the kidneys, renal insufficiency, diseases of the supporting apparatus, such as rheumatism, the sense organs, such as cataracts, or make a significant contribution to the development of disease or else influence the course of the convalescence.

The long-term use of non-steroidal anti-inflammatory drugs (NSAIDs) is associated with a quite pronounced gastrotoxicity. In the case of longer-lasting treatments, it results relatively often in irritations of the gastric mucous membrane, in gastric bleeding as well in the formation of ulcers. NSAIDs are the second most common cause of gastric and duodenal ulcers. The bleeding that occurs can be life-threatening. This fact represents a significant problem, since in the case of neurodegenerative diseases, almost only longer-term treatments appear useful.

NSAIDs, such as ibuprofen, occupy prominent positions in statistics in pharmaceutical agent side effects. In a report in the New England Journal of Medicine on the side effects of NSAIDs, 16000 people die from necrosis every year in the U.S.A. (Wolfe et al. 1999).

As can be confirmed by literature, the toxicity of several ibuprofen derivatives is significantly less compared to ibuprofen (Lolli et al. 2001).

As mentioned above, the use of NSAIDs is an eminently advantageous and realistic possibility for treating degenerative diseases of the central nervous system. Substances that have an antioxidative effect, such as Vitamin E and others, also represent a promising approach, as already mentioned. Nevertheless, the effectiveness of the two treatment strategies is thus limited in that these active substances, in particular the NSAID, can overcome the blood-brain barriers and can get into the CNS only to a very limited extent.

A strategy for improving the passage of the blood-brain barrier is the formation of prodrugs, i.e., compounds that themselves have only a little or no biological activity. Only by metabolic processes are the actual active ingredients released, and they can then exert their action (Albert, 1958). The claimed compounds represent so-called “Carrier-Mutual Prodrugs,” i.e., NSAID and tocopherol can be regarded in each case as carriers of the other components according to the invention. To be able to vary the properties of the compounds to a greater extent, derivatives also according to the invention were represented supplementing the two-component prodrugs with a spacer between the active ingredient groups and thus a three-component prodrug. Not only resorption and CNS accessibility, but also the extent and speed of hydrolysis can be modified by the spacer.

Chemical compounds of general structure I as racemates, enantiomers as well as diastereomers as well as in the form of their physiologically harmless salts and solvates, especially hydrates as well as addition compounds with alcohols, are subjects of the invention. These compounds are distinguished in that a pharmaceutical active ingredient “R-A” as well as tocopherol “Toc” therein optionally are linked to one another via one one or more spacers B according to formula 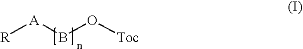
via an oxygen atom.

Radical R refers to the unchanged portion of the variable pharmaceutical active ingredient molecule. The structure R-A-OH (if the partial structure ‘A’ can be shown as C═X or SOₘ) or R-AH (for A=X) can thus be attributed to the pharmaceutical active ingredient that is used.

R symbolizes in particular the acyl radicals of NSAID, such as acetylsalicylic acid, diclofenic acid, ibuprofen, indomethacin, ketoprofen, mefenamic acid, naproxen as well as derivatives thereof, especially reduction products of indomethacin, whereby the CON partial structure is replaced formally by —CH₂N, as well as ketoprofen, whereby the keto-carbonyl group is replaced formally by —CH(OH)— or by —CH₂—.

The abbreviation Toc refers to a tocopheryl radical, in which R′, R″ and R′″ mean H or methyl. As can be seen from the following formula, three asymmetrical C atoms are present here, consequently there are eight diastereomeric forms. All diastereomers as well as mixtures thereof according to the invention are to be claimed.

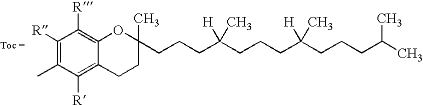

The invention comprises the chemical compounds of general formula I with respect to all possible racemates, enantiomers as well as diastereomers. If an acidic or basic partial structure is present in the compounds of formula I (e.g., derivatives of mefenamic acid or diclofenic acid), their physiologically harmless salts are also subjects of this invention. In addition, the invention also comprises solvates, especially hydrates and alcohol addition compounds, compounds I as well as their physiologically harmless salts.

For all radicals that can occur in several places, such as substituent ‘X,’ it holds true that their meanings are independent of one another:

A stands for C═X, SOₘ, X or CH₂, whereby

X represents O, S or NR¹ (with n≧1) or S or NR¹ (with n=0),

B refers to the grouping X—R²—Y,
in which Y stands for C═X, SOₘ or C(XR³)R⁴,

n means 0, 1, 2, 3, 4, 5 or 6 and is preferably 0, 1, 2 or 3,

m stands for 1 or 2 (preferably)

R¹ stands for H, a C₁-C₁₀-alkyl radical (preferably a C₁-C₆-alkyl radical), an aryl radical, a Het radical or an aryl or Het radical that is bonded via a C₁-C₆-spacer (preferably C₁-C₃).

R² stands for an alkylene, arylene or Het spacer or, however, combinations thereof, whereby the latter are linked to one another either directly or else via the function that is defined above as A or via the grouping X₀-A-Xₚ. The spacers can be defined analogously to the radicals “alkyl,” “aryl” and “Het.”

o and p stand for 0, 1 or 2; they can be the same or different.

R³ and R⁴ stand for H, a C₁-C₁₀-alkyl radical (preferably a C₁-C₆-alkyl radical), an aryl radical, a Het radical or an aryl or Het radical that is bonded via a C₁-C₆-spacer (preferably C₁-C₃).

Alkyl radicals are defined as hydrocarbons that are unbranched, branched or cyclic, saturated or partially unsaturated with double and/or triple bonds, unsubstituted or substituted in at least one place, preferably with F, Cl, Br, CN, NO₂, NR⁶R⁷, CHO, SOₘalkyl, OR⁶, COR⁶, COOR⁶, COCOR⁶, or CONR⁶R⁷. If the alkyl radical contains more than one substituent, the latter can be the same or different. The alkyl radicals are preferably methyl, ethyl, propyl, isopropyl, butyl, isobutyl, tert-butyl, cyclopropyl, cyclopentyl or cyclohexyl.

Aryl radical stands for an unsubstituted phenyl radical or a phenyl radical that is substituted in at least one place, preferably with F, Cl, Br, CN, alkyl, CF₃, NO₂, NR⁶R⁷, CHO, SOₘalkyl, OH, OR⁶, COR⁶, COOR⁶, COCOR⁶, CONR⁶R⁷, CSNR⁶R⁷ or aryl or that is Het-substituted. The phenyl radical can be condensed with additional cycles.

The Het radical refers to a saturated, unsaturated or aromatic monocyclic or bicyclic heterocyclic compound with 5 to 10 ring members, with at least one heteroatom, preferably nitrogen, oxygen and/or sulfur, and which optionally is provided with a fused carbocyclic compound or heterocyclic compound.

R⁶ and R⁷ stand for H, a C₁-C₁₀-alkyl radical, preferably a C₁-C₆-alkyl radical, an aryl radical, a heteroaryl radical or an aryl or heteroaryl radical that is bonded via a C₁-C₆ spacer, preferably C₁-C₃.

In addition, the invention relates to a process for the production of the chemical compounds of general formula (I).

For the production of the compounds of type I-1 according to the invention with A=CO and n=0—these are O-acyltocopherols in these compounds—different methods can be used. In principle, two variants are available; on the one hand, the esterification of the tocopherol by direct reaction with a corresponding free carboxylic acid and, on the other hand, the acylation reaction of the phenol derivative of tocopherol with an activated carboxylic acid derivative. Below, these variants are presented in more detail. 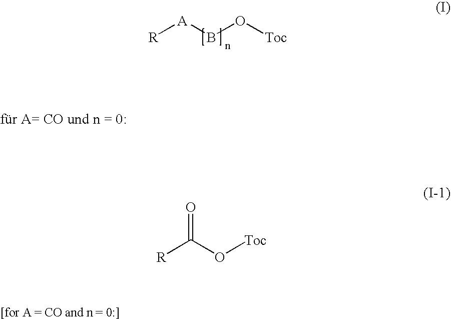
A) Starting from the free carboxylic acid, the synthesis of the claimed compounds of Type I-1 can be carried out by reaction with a tocopherol.

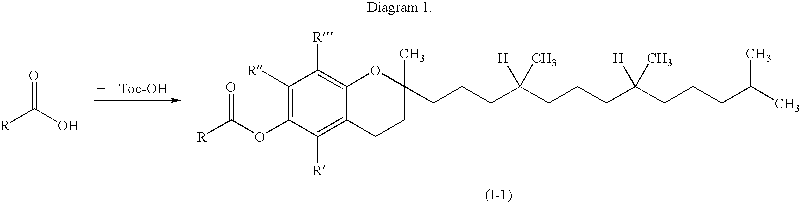

For example, the following procedures are suitable for these variants:

A 1) The reaction is carried out in the presence of an organic condensing agent. Examples of suitable condensing agents are dicyclohexylcarbodiimide (DCC), carbonyldiimidazole (CDI), thionylduimidazole (ThDI) and 1-hydroxy-1H-benzotriazole (HBT);

A 2) The reaction is carried out in the presence of an inorganic condensing agent, for example an inorganic anhydride, such as phosphorus pentoxide or an inorganic acid halide, such as phosphorus oxychloride.

A 3) The reaction is carried out as an acid-catalyzed condensation reaction. For this purpose, the addition of catalytic amounts of a non-oxidizing, strong acid is suitable. This can be both of an inorganic nature (e.g., concentrated sulfuric acid) and of an organic nature (e.g., benzenesulfonic acid or toluenesulfonic acid). In this process, the continuous removal of water that is produced in the condensation reaction, for example by azeotropic distillation and separation with the aid of a water separator, has proven its value.

Implementation can be performed either in an inert solvent or solvent mixture optionally, however, even in the absence of a solvent. Because of the reactivity of the components as well as the solvent that is used, the reaction is carried out at −10 to 250° C., whereby the reaction is carried out according to A) or usually already at low temperature (in general at room temperature), and usually relatively drastic conditions are necessary for the esterification according to B) or C): this applies primarily for variant C), since here a continuous distillative separation of the water is necessary.

B) Because of the generally relatively low reactivity of carboxylic acids, usually carboxylic acid derivatives are used in the acylation reactions. The methods that are claimed below are referred to in that a derivative of the carboxylic acid with higher reactivity is used. This activated compound can also be formed in situ by the derivative not being isolated from the reaction mixture but rather being further reacted directly with the nucleophile tocopherol to form the claimed compounds of structure I.

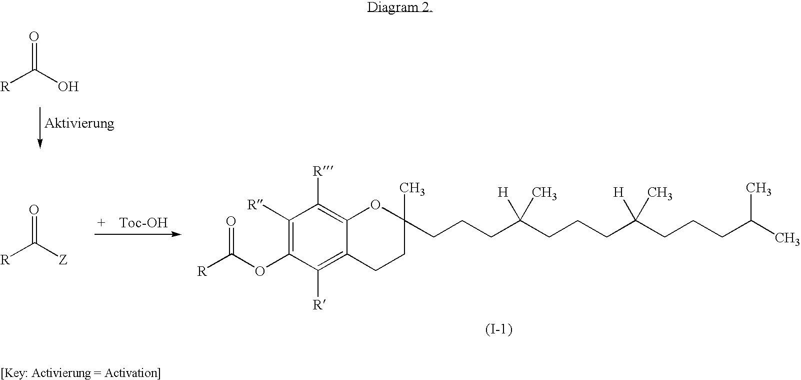

Examples of the process for the production of the compounds of type I according to the invention with A=CO and n=0 are cited below:
B1) The ester synthesis that originates from an acid halide—usually an acid chloride or acid bromide—and that is performed in the presence of a base (usually triethylamine, triethylamine/4-dimethylaminopyridine, pyridine, pyridine/4-dimethylaminopyridine, N-methyl-morpholine, Hünig base) has proven its value especially well. For the production of acid halide, different reagents of inorganic nature (e.g., thionyl chloride) or organic nature (e.g., oxalyl chloride, or 2,4,6-trichloro-1,3,5-triazine) can be used, whereby the activation by means of 2,4,6-trichloro-1,3,5-triazine is in particular also very good for the synthesis of esters in the single-pot process, in which primarily the acid chloride is produced and then directly further reacted.
B2) As an alternative, the target compounds can be produced by acylation of tocopherol by means of an acid anhydride. This reaction is optionally carried out with the addition of a base, for example pyridine. As acylating reagents according to these methods, both pure anhydrides of the pharmaceutical substance as well as mixed anhydrides, preferably anhydrides from the pharmaceutical substance and carboxylic acid monoester, are suitable.
B3) Production of the claimed compounds of type I by reesterification: in this process, an easily cleavable ester (for example methyl ester or ethyl ester, but also thioester) is reacted with tocopherol.
B4) An alternative technique is distinguished in that first the phenolic function is deprotonated in tocopherol and the phenolate that is produced is then converted into the corresponding target compound by reaction with an activated acid derivative (in particular acid chloride or acid anhydride).

As indicated above, the previous statements apply for compounds with X═CO and n=0, i.e., for esters. Derivatives in which the radical ‘A’ represents one of the other functions are made accessible analogously to processes that are generally known, as they are to be found in standard works, for example in ‘Houben-Weyl: Methoden der Organischen Chemie [Methods of Organic Chemistry],’ Georg Thieme Verlag, Stuttgart.

In addition, it is possible subsequently to derivatize the previously described carboxylic acid esters. This includes in particular the reduction of the —COOToc-partial structure to —CH₂Otoc. This subsequent derivatization thus represents a variant of the direct etherification. The compound R—CH₂OH can produce, for example, a reduced form of a biologically active carboxylic acid.

The compounds of structure I according to the invention with n≧1 is carried out formally from the components active ingredients (in structure I, the unchanged portion is referred to as R), spacer (B) and tocopherol. The linkage of these components can be carried out in different ways and in various sequences. The necessary reaction steps are dependent on these substituents in compounds of structure I—in particular on ‘A’ and spacer ‘B.’ These reaction steps comprise the processes of oxidation, reduction, ether cleavage, acylation, alkylation, etc., that are well-known to one skilled in the art. The use of protective groups, in particular standard hydroxyl and amino protective groups, can also be necessary; the use of the p-methoxybenzyl group as a protective group, for example a hydroxyl function in combination with the benzyl group as an amino protective group, is especially preferred.

In the diagram below, several variants are shown for the production of compounds I. These variants are used only for illustration, however, and do not limit the scope of the invention to the latter.

In general, it should be noted that in the reactions, bifunctional derivatives are frequently used. It follows from the above that the conditions in any case are selected such that the corresponding components are not reacted with themselves, i.e., neither intermolecularly nor intramolecularly, or that only one of the existing functions of the bifunctional components is derivatized. It can be achieved, on the one hand, that reaction conditions, such as reaction temperature, solvent, auxiliary base or auxiliary acid, catalyst and/or reaction time, that are as favorable as possible are carried out by selection, or by using suitable protective groups. The use of protective group techniques can be carried out both with isolated product and in the reaction mixture; the same also holds true for the cleavage of the protective groups. In the reaction diagram, no protective groups are indicated for the sake of clarity. In many cases, however, this technique cannot be eliminated. It is known to one skilled in the art when protective groups are required and with what protective group the best results can be achieved with the given formulation of the problem. An example of working with protective groups is also cited in the synthesis examples. In addition, it should be noted that optionally primarily an activation of the indicated component(s) is necessary. In which cases this is necessary and in what way an activation can be carried out are known to one skilled in the art. The activation is possible, if necessary, with the aid of known conversion reactions.

Diagram 3 below shows various variants for the production of such ‘three-component products’ (compounds with spacer components).

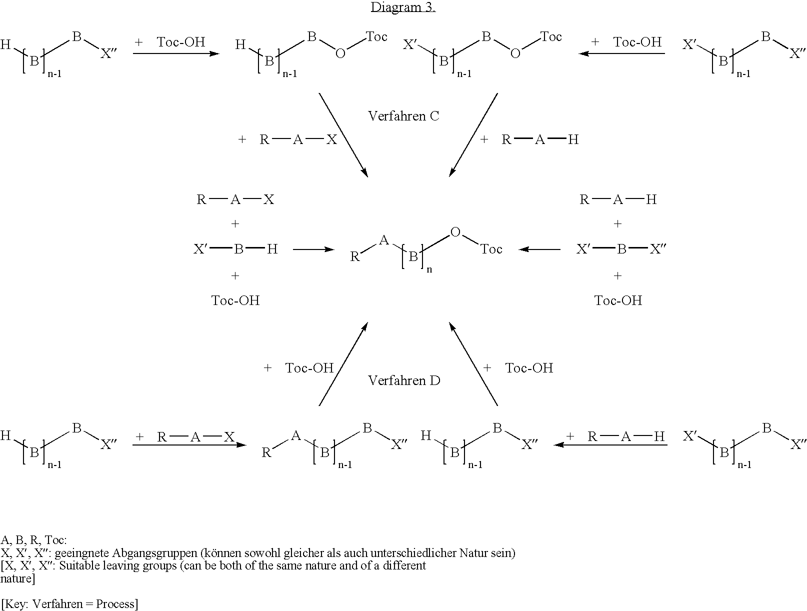

A variant of the multistage synthesis of compounds of type I with n≧1 represents—as the diagram shows—the primary formation of the spacer-tocopherol adducts. The subsequent reaction with the active ingredient results in the introduction of radical R and thus in compounds I according to the invention. In addition, in this variant C, it can then be distinguished which of the components—active ingredient to be introduced or spacer-spacer components—the leaving group carries and which of the used compounds the acceptor function takes over (see processes C1 and C2). An alternative procedure can be found in variant D. In this case, first active ingredients and spacers are linked to one another and ultimately only reacted with tocopherol. Another differentiation in methods D1 and D2 is carried out analogously to process C that is described above. The reaction steps that are necessary for linkage of the individual components are dependent on the existing substituents in the compounds involved, whereby primarily acylation reactions and alkylation reactions play an essential role. The reaction scheme can be carried out analogously to the methods that are described in the literature. The latter are known to one skilled in the art and do not need any further statement.

The reaction scheme can also be carried out in the way that the two-component intermediate stages are formed only in situ and then, without isolation from the reaction mixture, can be further reacted. General assessments relative to suitability or preferability of one of the described synthesis variants is not possible. The selection of the suitable process rather arises, i.a., from the availability of the required starting materials or the access to the latter and the protective group techniques that are necessary in each case. The necessary starting materials are generally known or commercially available; unknown educts can be produced analogously to the known compounds.

The claimed compounds can also be synthesized directly from the three components under suitable reaction conditions, although here, i.a., lower yields of the desired compound result.

Based on compounds of structure 1-2, which are compounds of type I with A=CO, n=1 and B═O—CH₂CO, key steps of the synthesis of the claimed compounds below are to be explained in more detail. The substance class selected for this purpose or the cited examples are used, however, only for illustration, without limiting the invention to their scope. The subsequent statements can also be assigned to the substituted derivatives directly or with minor modifications.

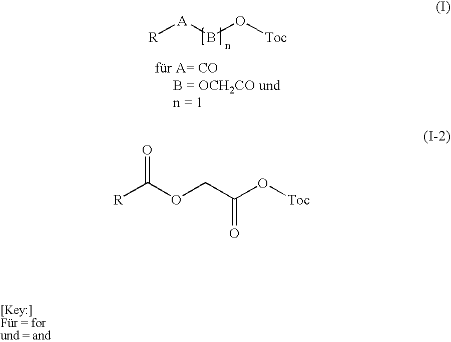

As a spacer, a glycolic acid component is present in compounds 1-2; this component can be introduced in a different way corresponding to the above-discussed variants (protective groups are not indicated at this point), for example by
Reaction of activated glycolic acid or of free glycolic acid according to one of the processes, described under A1-A3, with tocopherol and subsequent O-acylation by reaction with the active ingredient R—COOH or an activated derivative thereof (=variant C1)
Primary reaction of α-haloacetic acid or an activated derivative, such as, for example, α-haloacetyl halide; because of the varying high reactivity of the two halogen atoms, only an acylation with tocopherol and subsequent alkylation of the carboxylic acid function of the active ingredient molecule are carried out here under suitable reaction conditions by reaction with the free active ingredient (R—COOH) in the presence of a base (=variant C2).
Primary acylation of the active ingredient molecule by reaction of R—COOH or an activated derivative with glycolic acid and subsequent O-acylation of tocopherol under suitable reaction conditions or after prior activation (variant D1)
Esterification of the active ingredient by alkylation reaction with α-haloacetic acid or a derivative that is not activated, however, and subsequent acylation of the tocopherol (variant D2). Here, i.a., an activation precedes the second step.

Starting from the educts that are used in C or D, a direct reaction of all components can also be carried out.

The variants that are discussed here are shown in Diagram 4 below.

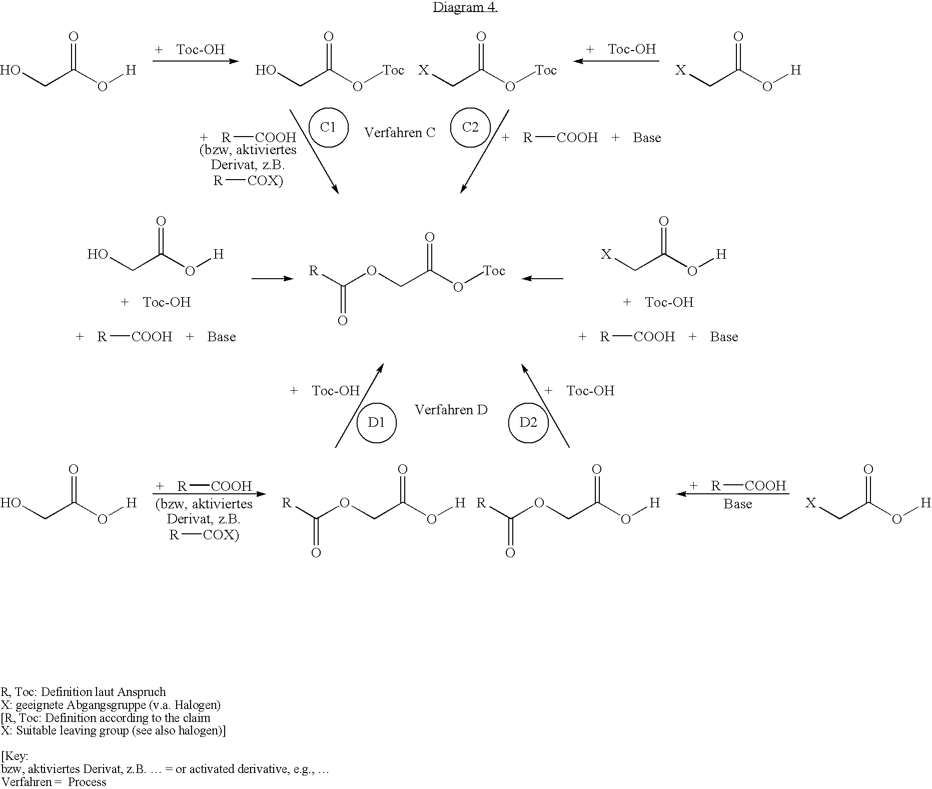

Below and under Experiments, one of these synthesis strategies (D2)—this time taking into consideration protective groups—is explained in more detail based on the examples (see also Diagram 5). No preference of the process compared to one of the other described methods or else analogous processes can necessarily be deduced from this, however.

In the case of the build-up of these double carboxylic acid esters according to the method that is presented under Experiments, the first step is the linkage of the acidic active ingredient with the spacer. The production of these active ingredient-glycolic acid esters is possible by, for example, the alkylation reaction. While the use of free α-haloacetic acid can lead to the formation of undesirable by-products, the use of a compound with protected carboxylic acid function has proven its value. With respect to the additional synthesis steps, a protective group can be selected that can be cleaved under very mild reaction conditions. For example, a suitable benzyl ester fulfills this requirement, since such esters, experience shows, can be cleaved selectively.

For the production of the O-acylated glycolic acid benzyl ester, a solution of the respective active ingredient, for example the corresponding NSAID, is mixed with an auxiliary base, and then the carboxylate anion that is formed is converted by reaction with bromoacetic acid benzyl ester into the corresponding O-acylated glycolic acid ester.

After the hydrogenolytic cleavage of the protective group and subsequent activation of the carboxylic acid function, for example by conversion into the corresponding acid chloride, which is possible, i.a., by reaction of the free carboxylic acid with 2,4,6-trichloro-1,3,5-triaizne and N-methylmorpholine, the three-component prodrugs that are claimed here can be obtained by esterification with tocopherol.

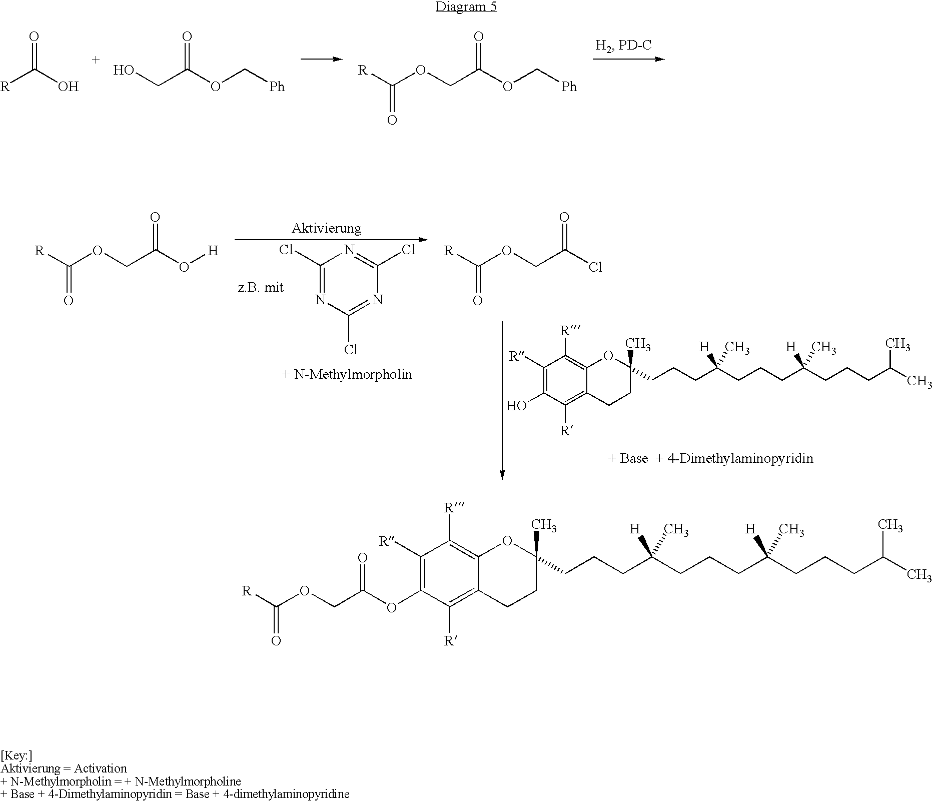

Depending on the substitution pattern, the compounds of type I that are described as well as their precursors can be further functionalized according to processes that are known in the literature. These derivatizations comprise the processes of oxidation, reduction, ether cleavage, acylations, alkylations, etc., that are well-known to one skilled in the art.

In addition to the carrier-linked prodrugs, compounds that can be regarded as bioprecursors, i.e., that can be converted by non-hydrolytic metabolization into the corresponding two- or three-component prodrugs or else directly into active ingredients, are also claimed. The compounds that are cited below are only examples of this type of combined bioprecursor-carrier prodrug; the scope of the invention, however, is not to be limited to this scope.

The keto function that is present in the NSAID ketoprofen is an option for, for example, derivatization; by reduction, the latter can be converted into the corresponding alcohol or else into a methylene grouping. In the organism, the benzophenone structure can be further produced by oxidation of the benzhydrole or diphenylmethane partial structure.

The production of such combined bioprecursor-carrier prodrugs is possible by reduction reaction, which can be carried out in different stages. Since the synthesis sequence that is described contains a hydrogenation step, it is advantageous to perform the reduction of the ketone in this stage.

[image]

Examples of Strategies for the Synthesis of Novel Two- and Three-Component Prodrugs

The invention is explained in more detail below based on the embodiments for implementing the invention. These examples are used only for illustration, without, however the scope of the invention being limited to this scope.

Process for the Synthesis of Two-Component Prodrugs in the Example of NSAID-Tocopherol-Esters

Single-Pot Process:

1.3 equivalents (2.80-4.85 mmol) of the respective carboxylic acid is suspended in 40 ml of acetonitrile, mixed with 0.33 equivalent (0.72-1.24 mmol) of 2,4,6-trichloro-1,3,5-triazine as well as 1.1 equivalents (2.37-4.10 mmol) of N-methylmorpholine and stirred for 2.5 hours at room temperature. After 1.0 equivalent (2.15-3,73 mmol) of α-tocopherol (dissolved in about 5 ml of absolute dichloromethane) and catalytic amounts of 4-dimethylaminopyridine are added, the reaction batch is heated to 45-50° C. and stirred at this temperature until conversion is as complete as possible (reaction monitoring by means of thin-layer chromatogram=TLC).

For working-up, the solvent is distilled off in a vacuum, and the residue is taken up in dichloromethane. The organic phase is washed with 2M hydrochloric acid and saturated sodium bicarbonate solution, then washed neutral with water and pre-dried with saturated sodium chloride solution; after drying on anhydrous sodium sulfate, the solvent is distilled off. The thus obtained crude product is then purified, for example, by means of column chromatography.

Two-Stage Synthesis:

One equivalent of the respective carboxylic acid is converted with the aid of one of the standard processes, i.e., for example by treatment with thionyl chloride or oxalyl chloride, into the corresponding carboxylic acid chloride. The crude product that is obtained after the reaction is completed can be used either directly or after purification (preferably by distillation) for acylation of the corresponding nucleophile (e.g., α-tocopherol). For acylation, the activated carboxylic acid and the nucleophile are brought to reaction in approximately equivalent amounts in an inert solvent (for example dichloromethane, tetrahydrofuran, dioxane, dimethylformamide, acetonitrile, or the like) in the presence of an auxiliary base (preferably triethylamine or pyridine)—optionally after an acylation catalyst (preferably 4-dimethylaminopyridine) is added.

Below, some examples of compounds that can be produced in this way are cited:

### EXAMPLE 1

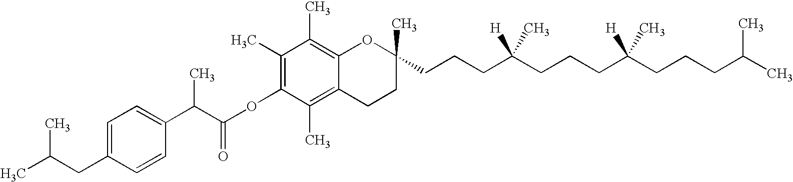

| | | |
|---|---|---|
| Active ingredient components: | NSAID: | Dexibuprofen |
| | Antioxidant: | α-Tocopherol |

| Reaction time 43 hours | |
|---|---|
| Appearance: | Light-yellow viscous oil |
| Purification | Column chromatography: Stationary phase: Silica gel, Mobile phase: |
| | Dichloromethane/Petroleum ether (Ratio: 10/1) |

| Elementary analysis: | C | H |
|---|---|---|
| Relative to C₄₂H₆₆O₃ | Cld. 81.50% | 10.75% |
| (618.99) | Fnd. 81.31% | 10.95% |

IR (KBr) 1751 cm⁻¹
MS (CI) 619.5 (M+1)⁺
¹H-NMR (CDCl₃)

| | | Relative | |
|---|---|---|---|
| δ Values [ppm] | Multiplicity | Intensity | Identification |
| 7.35 | d, J=8.0Hz | 2H | Phenyl-H |
| 7.12 | d, J=8.0Hz | 2H | |
| 3.98 | q, J=7.1Hz | 1H | Phenyl-CH—CH₃ |
| 2.52 | t, J=6.6Hz | 2H | 4-CH₂ (Chroman) |
| 2.47 | d, J=7.2Hz | 2H | —CH₂—CH(CH₃)₂ |
| 2.03 | S | 3H | 5-CH₃, 7-CH₃, 8-CH₃, |
| 1.67 | S | 3H | |
| 1.64 | S | 3H | |
| 1.23 | S | 3H | 2-CH₃ |
| 1.96-1.02 | M | 27H | 3-CH₂ (Chroman), |
| | | | Phenyl-CH—CH₃, |
| | | | —CH₂—CH(CH₃)₂, CH, |
| | | | CH₂ |
| 0.91-0.82 | M | 18H | 4 × CH₃ |
| | | | (Tocopherol Side Chain) |
| | | | —CH₂—CH(CH₃)₂ |

### EXAMPLE 2

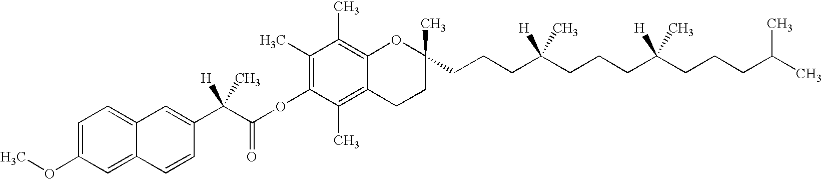

| | | |
|---|---|---|
| Active ingrednt components: | NSAID: | Naproxen |
| | Antioxidant: | α-Tocopherol |

| Reaction time 40 hours | |
|---|---|
| Appearance: | Yellow viscous oil |
| Purification | Column chromatography: Stationary phase: Silica gel, Mobile phase: |
| | Dichlorromethane/Petroleum eether (Ratio: 4/1) |

| Elementary Analysis | C | H |
|---|---|---|
| Relative to C₄₃H₆₂O₄ | Cld. 80.33% | 9.72% |
| (642.97) | Fnd. 80.27% | 10.02% |

IR (KBr) 1749 cm⁻¹
MS (CI) 643.5 (M+1)⁺
¹H-NMR (CDCl₃)

| | | Relative | |
|---|---|---|---|
| δ Values [ppm] | Multiplicity | Intensity | Identification |
| 7.82-7.70 | M | 3H | Naphthyl-H |
| 7.55 | dd, J=8.4Hz; | | |
| | J=1.8Hz | 1H | |
| 7.18-7.13 | M | 2H | |
| 4.14 | q, J=7.1Hz | 1H | Phenyl-CH—CH₃ |
| 3.92 | S | 3H | —OCH₃ |
| 2.51 | ‘t’ | 2H | 4-CH₂ (Chroman) |
| 2.14-1.00 | M | 26H | 3-CH₂ (Chroman), |
| | | | Phenyl-CH—CH₃, CH, CH₂ |
| 2.02 | S | 3H | 5-CH₃, 7-CH₃, 8-CH₃, |
| 1.76 | S | 3H | |
| 1.72 | S | 3H | |
| 1.20 | S | 3H | 2-CH₃ (Chroman) |
| 0.88-0.82 | M | 12H | 4 × CH₃ |
| | | | (Tocopherol Side Chain) |

### EXAMPLE 3

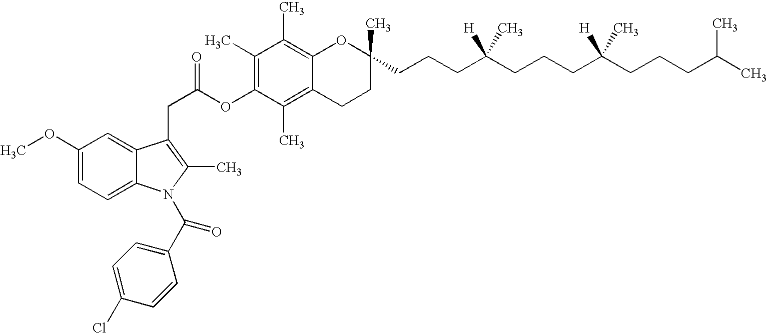

| | | |
|---|---|---|
| Active ingredient components: | NSAID: | Indomethacin |
| | Antioxidant: | α-Tocopherol |

| | |
|---|---|
| Reaction time | 40 hours |

| | |
|---|---|
| Appearance: | Light-yellow viscous oil |
| Purification | Column chromatography: Stationary phase: Silica gel, Mobile phase: Dichloromethane/Petroleum ether (Ratio: 10/1) |

| Elementary Analysis | | C | H | N |
|---|---|---|---|---|
| Relative to C₄₈H₆₄ClNO₅ | Cld. | 72.88% | 8.18% | 1.76% |
| × 0.3 CH₂Cl₂ | Fnd. | 72.89% | 8.25% | 2.18% |

| | |
|---|---|
| IR (KBr) | 1752, 1686 cm⁻¹ |
| MS(CI) | 770.4 (M + 1)⁺ |
| ¹H-NMR (CDCl₃) | |

| δ Values | | Relative | |
|---|---|---|---|
| [ppm] | Multiplicity | Intensity | Identification |
| 7.68-7.61 | m | 2H | Phenyl-H |
| 7.49-7.43 | m | 2H | |
| 7.08 | d, J=2.6Hz | 1H | Indole-H4 |
| 6.91 | d, J=9.1Hz | 1H | Indole-H7 |
| 6.68 | dd, J=9.1Hz; J=2.6Hz | 1H | Indole-H6 |
| 3.93 | s | 2H | Indole-CH₂—COOR |
| 3.82 | s | 3H | —OCH₃ |
| 2.54 | t, J=6.6Hz | 2H | 4-CH₂ (Chroman) |
| 2.45 | s | 3H | Indole-2-CH₃ |
| 2.05 | s | 3H | 5-CH₃, 7-CH₃, 8-CH₃, |
| 1.90 | s | 3H | |
| 1.85 | s | 3H | |
| 1.83-1.67 | m | 2H | 3-CH₂ (Chroman) |
| 1.62-1.02 | m | 21H | CH, CH₂ |
| 1.21 | s | 3H | 2-CH₃ (Chroman) |
| 0.88-0.83 | m | 12H | 4 × CH₃ |
| | | | (Tocopherol Side |
| | | | Chain) |

### EXAMPLE 4

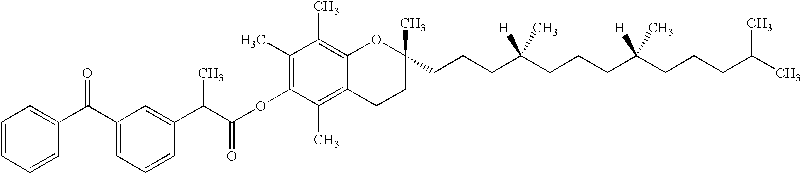

| | | |
|---|---|---|
| Active ingredient components: | NSAID: | Ketoprofen |
| | Antioxidant: | α-Tocopherol |
| Reaction time 15 hours | | |

| | |
|---|---|
| Appearance: | Light-yellow viscous oil |
| Purification | Column chromatography: Stationary phase: Silica gel, Mobile phase: Petroleum ether/Diethyl ether (Ratio: 3/1) |

| Elementary Analysis | | C | H |
|---|---|---|---|
| Relative to C₄₅H₆₂O₄ | Cld. | 78.57% | 9.11% |
| × 0.3 CH₂Cl₂ | Fnd. | 78.57% | 9.37% |

| | |
|---|---|
| IR (KBr) | 1750, 1662 cm⁻¹ |
| MS(CI) | 667.4 (M + 1)⁺ |
| ¹H-NMR (CDCl₃) | |

| | | Relative | |
|---|---|---|---|
| δ Values [ppm] | Multiplicity | Intensity | Identification |
| 7.91-7.42 | m | 9H | Phenyl-H |
| 4.10 | q, J=7.2Hz | 1H | Phenyl-CH—CH₃ |
| 2.54 | t, J=6.6Hz | 2H | 4-CH₂ (Chroman) |
| 2.05 | s | 3H | 5-CH₃, 7-CH₃, 8-CH₃, |
| 1.73 | s | 3H | |
| 1.69 | s | 3H | |
| 1.86-1.08 | m | 26H | 3-CH₂ (Chroman), |
| | | | Phenyl-CH—CH₃, CH, CH₂ |
| 1.21 | s | 3H | 2-CH₃ (Chroman) |
| 0.91-0.82 | m | 12H | 4 × CH₃ |
| | | | (Tocopherol Side Chain) |

Process for the Synthesis of Three-Component Prodrups in the Example of NSAID-Glycolic Acid-Tocopherol Esters

Production of Benzyl Esters

One equivalent (4.5-9.0 mmol) of the respective carboxylic acid is suspended in 20 ml of absolute N,N-dimethylformamide, and mixed with 1.5 equivalents of potassium carbonate (6.75-13.5 mmol) and a spatula-tip full of sodium iodide. Then, while being stirred constantly and being cooled with ice, 5.0 equivalents (22.5-45.0 mmol) of bromoacetic acid benzyl ester (dissolved in 10 ml of absolute N,N-dimethylformamide) is added in drops within the course of one hour. The batch is stirred until conversion is as complete as possible; the reaction time is 16-18 hours (reaction monitoring by means of TLC).

After the reaction is completed, the reaction solution is added to about 50 ml of ice water: thereupon a precipitate forms, the latter is filtered off by suction and washed several times with water and petroleum ether. The crude product that is obtained is recrystallized from diisopropyl ether; the pure substance that is obtained is dried in the desiccator until a constant weight is reached.

If a precipitate is formed, the aqueous phase is exhaustively extracted with dichloromethane. The combined organic phases are washed several times with saturated sodium chloride solution and dried on anhydrous sodium sulfate. Then, the solvent is distilled off. To remove this N,N-dimethylformamide—this has a disturbing effect in column chromatography—the residue is taken up in ether, and the ether phase is washed with water as well as saturated sodium chloride solution. After drying on anhydrous sodium sulfate, the solvent is distilled off, and the crude product that is obtained is purified by means of column chromatography (silica gel, LM: 1) PE (for eluting bromoacetic acid benzyl ester), 2) ether (for eluting the product).

Cleavage of the Protective Group by Means of Hydrogenation

One equivalent (4.92-8.17 mmol) of the respectively benzyl ester derivative is dissolved in 150 ml of tetrahydrofuran, covered for three minutes with nitrogen and mixed with 0.2 g of palladium on activated carbon per g of benzyl ester. At a pressure of at most 50 Psi, it is hydrogenated at room temperature while being shaken constantly (reaction time: 2.5-24 hours, the reaction is monitored by means of TLC).

After the reaction is completed, the solution is covered again with nitrogen, the catalyst is filtered off, and the solvent is distilled off in a vacuum. The crude product that is obtained is purified either by means of recrystallization from a suitable solvent (e.g., diisopropyl ether) or by column chromatography.

Activation and Esterification with Tocopherol

1.3 equivalents (1.50-3.98 mmol) of the respective glycolic acid derivative is suspended in 40 ml of acetonitrile, mixed with 0.33 equivalent (0.38-0.97 mmol) of 2,4,6-trichloro-1,3,5-triazine and 1.1 equivalents (1.27-3.20 mmol) of N-methylmorpholine, and stirred for 2.5-3 hours at room temperature. After 1.0 equivalent (1.15-2.91 mmol) of α-tocopherol is added (dissolved) in about 5 ml of absolute dichloromethane) and one spatula-tip full of 4-dimethylaminopyridine, the reaction batch is heated to 45-60° C. and stirred until the conversion is as complete as possible: the reaction time is 17-65.5 hours (reaction monitoring by means of TLC).

For working-up, the solvent is distilled off in a vacuum, the residue is taken up in ethyl acetate, the organic phase is washed with 2M hydrochloric acid and saturated sodium bicarbonate solution, neutralized with water and predried with saturated sodium chloride solution. After drying on anhydrous sodium sulfate, the solvent is completely distilled off, and the thus obtained crude product is purified by means of column chromatography.

### EXAMPLE 5

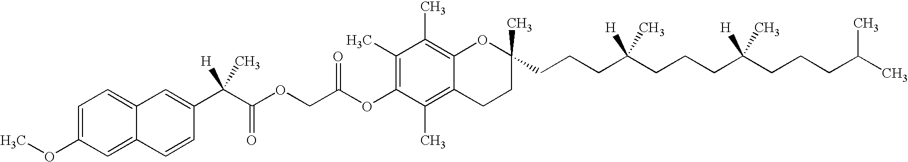

| | | |
|---|---|---|
| Active ingredient components: | NSAID: | Naproxen |
| | Spacer: | Glycolic acid |
| | Antioxidant: | α-Tocopherol |

| Reaction time 17 hours | |
|---|---|
| Appearance: | Dark-yellow viscous oil |
| Purification | Column chromatography: Stationary phase: Silica gel, Mobile phase: |
| | Petroleum Ether |

| Elementary analysis: | C | H |
|---|---|---|
| Relative to C₄₅H₆₄O₆ | Cld. 77.10% | 9.20% |
| (701.01) | Fnd. 77.34% | 8.93% |

IR (KBr) 1777, 1746 cm⁻¹
MS (CI) 701.3 (M+1)⁺
¹H-NMR (CDCl₃)

| δ Values | | Relative | |
|---|---|---|---|
| [ppm] | Multiplicity | Intensity | Identification |
| 7.69-7.63 | m | 3H | Naphthyl-H |
| 7.42 | dd, J=8.4Hz; J=2.0Hz | 1H | |
| 7.14-7.08 | m | 2H | |
| 4.92 | d, J=15.9Hz | 2H | —O—CH₂—COOR |
| 4.83 | d, J=15.9Hz | | |
| 4.01 | q, J=7.2Hz | 1H | Phenyl-CH—CH₃ |
| 3.90 | s | 3H | —OCH₃ |
| 2.56 | t, J=6.5Hz | 2H | 4-CH₂ (Chroman) |
| 2.07 | s | 3H | 5-CH₃, 7-CH₃, 8-CH₃, |
| 1.97 | s | 3H | |
| 1.92 | s | 3H | |
| 1.85-1.69 | m | 2H | 3-CH₂ (Chroman) |
| 1.64 | d, J=7.2Hz | 3H | Phenyl-CH—CH₃ |
| 1.59-1.08 | m | 21H | CH, CH₂ |
| 1.23 | s | 3H | 2-CH₃ (Chroman) |
| 0.88-0.83 | m | 12H | 4 × CH₃ |
| | | | (Tocopherol Side |
| | | | Chain) |

### EXAMPLE 6

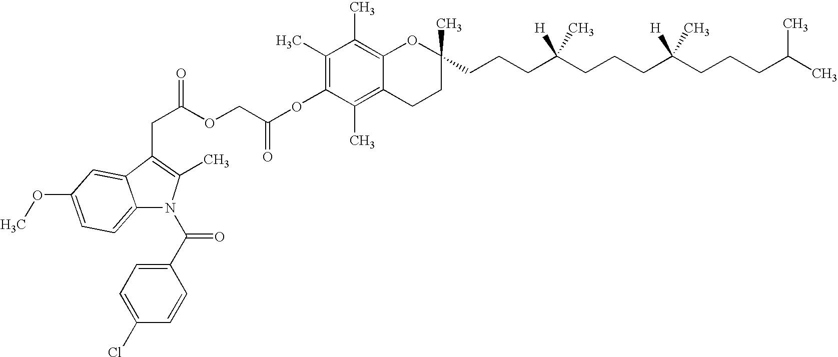

| | | |
|---|---|---|
| Active ingredient components: | NSAID: | Indomethacin |
| | Spacer: | Glycolic acid |
| | Antioxidant: | α-Tocopherol |

| Reaction time 24 hours | |
|---|---|
| Appearance: | Light-yellow foam resin |
| Purification | Column chromatography: Stationary phase: Silica gel, Mobile phase: |
| | Dichloromethane/Petroleum ether (Ratio: 10/1) |

| Elementary Analysis | C | H | N |
|---|---|---|---|
| Relative to C₅₀H₆₆ClNO₇ | Cld. 72.48% | 8.03% | 1.69% |
| (828.54) | Fnd. 72.30% | 7.91% | 1.81% |

IR (KBr) 1770, 1736, 1680 cm⁻¹
MS (CI) 828.3 (M+1)⁺
¹H-NMR (CDCl₃)

| δ-Values | | Relative | |
|---|---|---|---|
| [ppm] | Multiplicity | Intensity | Identification |
| 7.62-7.58 | m | 2H | Phenyl-H |
| 7.44-7.40 | m | 2H | |
| 6.97 | d, J=2.5Hz | 1H | Indole-H4 |
| 6.86 | d, J=9.1Hz | 1H | Indole-H7 |
| 6.65 | dd, J=9.1Hz; J=2.5Hz | 1H | Indole-H6 |
| 4.92 | s | 2H | —O—CH₂—COOR |
| 3.81 | s | 2H | Indole-CH₂—COOR |
| 3.78 | s | 3H | —OCH₃ |
| 2.56 | t, J=6.2Hz | 2H | 4-CH₂ (Chroman) |
| 2.37 | s | 3H | Indole-2-CH₃ |
| 2.07 | s | 3H | 5-CH₃, 7-CH₃, 8-CH₃, |
| 1.95 | s | 3H | |
| 1.91 | s | 3H | |
| 1.82-1.72 | m | 2H | 3-CH₂ (Chroman) |
| 1.59-1.06 | m | 21H | CH, CH₂ |
| 1.23 | s | 3H | 2-CH₃ (Chroman) |
| 0.88-0.83 | m | 12H | 4 × CH₃ |
| | | | (Tocopherol Side |
| | | | Chain) |

### EXAMPLE 7

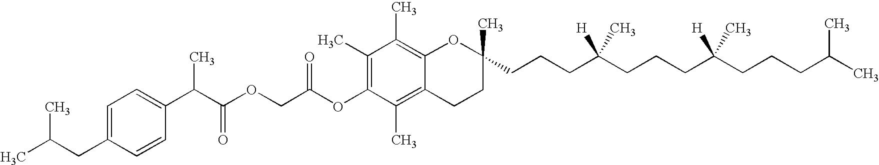

| | | |
|---|---|---|
| Active ingredient components: | NASID: | Dexibuprofen |
| | Spacer: | Glycolic acid |
| | Antioxidant: | α-Tocopherol |

| Reaction time 47 hours | |
|---|---|
| Appearance: | Light-yellow viscous oil |
| Purification | Column chromatography: Stationary phase: Silica gel, Mobile phase: |
| | Petroleum ether/Diethyl ether (Ratio: 4/1) |

| Elementary analysis | C | H |
|---|---|---|
| Relative to C₄₄H₆₈O₅ | Cld. 78.06% | 10.12% |
| (677.03) | Fnd. 77.84% | 9.94% |

IR (KBr) 1780, 1749 cm⁻¹
MS (CI) 677.5 (M+1)⁺
¹H-NMR (CDCl₃)

| | | Relative | |
|---|---|---|---|
| δ Values [ppm] | Multiplicity | Intensity | Identification |
| 7.24 | d, J=8.0Hz | 2H | Phenyl-H |
| 7.07 | d, J=8.0Hz | 2H | |
| 4.92 | d, J=15.9Hz | 2H | —O—CH₂—COOR |
| 4.81 | d, J=15.9Hz | | |
| 3.85 | q, J=7.3Hz | 1H | Phenyl-CH—CH₃ |
| 2.58 | t, J=6.8Hz | 2H | 4-CH₂ (Chroman) |
| 2.42 | d, J=7.0Hz | 2H | —CH₂—CH(CH₃)₂ |
| 2.08 | s | 3H | 5-CH₃, 7-CH₃, 8-CH₃, |
| 1.99 | s | 3H | |
| 1.95 | s | 3H | |
| 1.89-1.69 | m | 3H | 3-CH₂ (Chroman), |
| | | | —CH₂—CH(CH₃)₂ |
| 1.59-1.08 | m | 24H | Phenyl-CH—CH₃, CH, CH₂ |
| 1.23 | s | 3H | 2-CH₃ |
| 0.90-0.83 | m | 18H | 4 × CH₃ |
| | | | (Tocopherol Side Chain) |
| | | | —CH₂—CH(CH₃)₂ |

### EXAMPLE 8

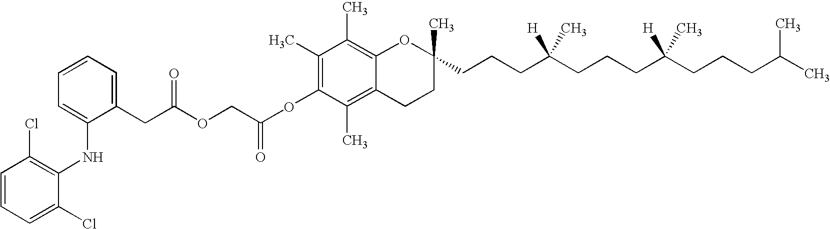

| | | |
|---|---|---|
| Active ingredient components: | NSAID: | Diclofenic acid |
| | Spacer: | Glycolic acid |
| | Antioxidant: | α-Tocopherol |
| Reaction time 34 hours | | |

| | |
|---|---|
| Appearance: | White-yellowish resin |
| Purification | Column chromatography: Stationary phase: Silica gel, Mobile phase: Dichloromethane/Petroleum ether (Ratio: 4/1) |

| Elementary Analysis | | C | H | N |
|---|---|---|---|---|
| Relative to C₄₅H₆₁Cl₂NO₅ (766.90) | Cld. | 70.48% | 8.02% | 1.83% |
| | Fnd. | 70.76% | 8.17% | 1.74% |

| | |
|---|---|
| IR (KBr) | 3368, 1763, 1745 cm⁻¹ |
| MS(CI) | 766.2 (M + 1)⁺ |
| ¹H-NMR (CDCl₃) | |

| δ Values | | Relative | |
|---|---|---|---|
| [ppm] | Multiplicity | Intensity | Identification |
| 7.34-7.24 | m | 3H | Phenyl-H |
| 7.42 | dt, J=7.7Hz, J=1.7Hz | 1H | |
| 7.01-6.92 | m | 2H | |
| 6.55 | d, J=8.0Hz | 1H | |
| 6.70 | ‘s’ (br) | 1H | NH |
| 4.95 | s | 2H | —O—CH₂—COOR |
| 3.96 | s | 2H | Ph—CH₂—COOR |
| 2.57 | t, J=6.8Hz | 2H | 4-CH₂ (Chroman) |
| 2.07 | s | 3H | 5-CH₃, 7-CH₃, 8-CH₃, |
| 2.00 | s | 3H | |
| 1.95 | s | 3H | |
| 1.85-1.69 | m | 2H | 3-CH₂ (Chroman) |
| 1.60-1.08 | m | 21H | CH, CH₂ |
| 1.22 | s | 3H | 2-CH₃ |
| 0.88-0.82 | m | 12H | 4 × CH₃ |
| | | | (Tocopherol Side |
| | | | Chain) |

### EXAMPLE 9

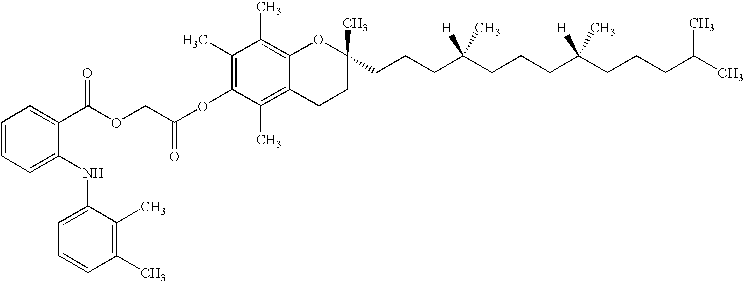

| | | |
|---|---|---|
| Active ingredient components: | NSAID: | Mefenamic acid |
| | Spacer: | Glycolic acid |
| | Antioxidant: | α-Tocopherol |
| Reaction time 66 hours | | |

| | |
|---|---|
| Appearance: | Light-yellow foam resin |
| Purification | Column chromatography: Stationary phase: Silica gel, Mobile phase: Dichloromethane/Petroleum ether |
| | (Ratio: 10/1) |

| Elementary Analysis | | C | H | N |
|---|---|---|---|---|
| Relative to C₄₆H₆₅NO₅ (712.03) | Cld. | 77.60% | 9.20% | 1.97% |
| | Fnd. | 77.34% | 8.97% | 2.12% |

| | |
|---|---|
| IR (KBr) | 3336, 1779, 1690 cm⁻¹ |
| MS(CI) | 712.3 (M + 1)⁺ |
| ¹H-NMR (CDCl₃) | |

| δ Values | | Relative | |
|---|---|---|---|
| [ppm] | Multiplicity | Intensity | Identification |
| 9.12 | s | 1H | NH |
| 8.08 | dd, J=8.0Hz, J=1.6Hz | 1H | Phenyl-H |
| 7.30-7.01 | m | 4H | |
| 6.73-6.63 | m | 2H | |
| 5.11 | s | 2H | —O—CH₂—COOR |
| 2.59 | t, J=6.5Hz | 2H | 4-CH₂ (Chroman) |
| 2.33 | s | 3H | 2 × Ph—CH₃ |
| 2.16 | s | 3H | |
| 2.09 | s | 3H | 5-CH₃, 7-CH₃, 8-CH₃, |
| 2.06 | s | 3H | |
| 2.02 | s | 3H | |
| 1.86-1.70 | m | 2H | 3-CH₂ (Chroman) |
| 1.58-1.02 | m | 21H | CH, CH₂ |
| 1.23 | s | 3H | 2-CH₃ |
| 0.88-0.82 | m | 12H | 4 × CH₃ |
| | | | (Tocopherol Side |
| | | | Chain) |

Examples of Combined Bioprecursor-Carrier Prodrugs:

The reduction of the keto function is carried out within the scope of the cleavage of the benzyl protective group by reaction with hydrogen at 50 Psi in the presence of a suitable catalyst; after the necessary amount of hydrogen is taken up, the reaction is halted. Then, the corresponding O-acylated glycolic acid is activated and reacted with tocopherol.

### EXAMPLE 10

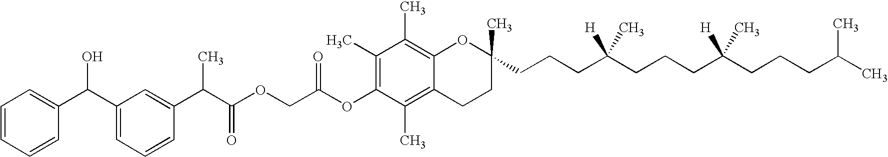

| | | |
|---|---|---|
| Active ingredient components: | NSAID: | (C═O)-partially reduced Ketoprofen |
| | Spacer: | Glycolic acid |
| | Antioxidant: | α-Tocopherol |

| Reaction time 60 hours | |
|---|---|
| Appearance: | Light-yellow viscous oil |
| Purification | Column Chromatography: Stationary phase: Silica gel, Mobile phase: |
| | Dichloromethane/Petroleum ether (Ratio: 4/1) |

| Elementary Analysis | C | H |
|---|---|---|
| Relative to C₄₇H₆₆O₆ | Cld. 76.20% | 9.00% |
| ×0.2 CH₂Cl₂ | Fnd. 76.35% | 8.83% |

IR (KBr) 3428, 1775, 1746 cm⁻¹
MS (CI) 727.6 (M+1)⁺
¹H-NMR (CDCl₃)

| | | Relative | |
|---|---|---|---|
| δ Values [ppm] | Multiplicity | Intensity | Identification |
| 7.36-7.17 | M | 9H | Phenyl-H |
| 5.69 | S | 1H | CH—OH |
| 4.82 | s | 2H | —O—CH₂—COOR |
| 3.84 | q, J=7.0Hz | 1H | Phenyl-CH—CH₃ |
| 2.56 | t, J=6.6Hz | 2H | 4-CH₂ (Chroman) |
| 2.45 | s (br) | 1H | CH—OH |
| | | | (D₂O-exchangeable) |
| 2.08 | s | 3H | 5-CH₃, 7-CH₃, 8-CH₃, |
| 1.96 | s | 3H | |
| 1.93 | s | 3H | |
| 1.84-1.67 | m | 2H | 3-CH₂ (Chroman) |
| 1.52 | d, J=7.0Hz | 3H | Phenyl-CH—CH₃ |
| 1.60-1.09 | m | 21H | CH, CH₂ |
| 1.18 | s | 3H | 2-CH₃ (Chroman) |
| 0.88-0.83 | m | 12H | 4 × CH₃ |
| | | | (Tocopherol Side Chain) |

### EXAMPLE 11

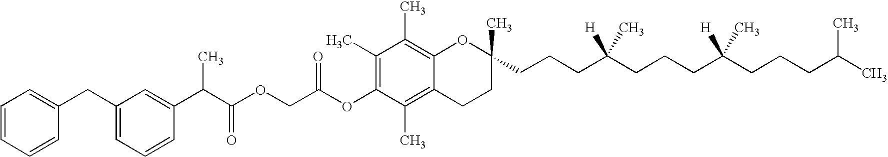

| | | |
|---|---|---|
| Active ingredient components: | NSAID: | (C═O)-reduced Ketoprofen |
| | Spacer: | Glycolic acid |
| | Antioxidant: | α-Tocopherol |
| Reaction time 60 hours | | |
| Appearance: | Light-yellow viscous oil | |
| Purification | Column chromatography: Stationary phase: Silica gel, Mobile phase: | |
| | Dichloromethane/Petroleum ether (Ratio:4/1) | |

| Elementary Analysis | | | C | H |
|---|---|---|---|---|
| Relative to | Cld. | 78.60% | 9.37% | |
| C₄₇H₆₆O₅ × 0.4 H₂O | Fnd. | 78.58% | 9.11% | |

IR (KBr) 1779, 1748 cm⁻¹
MS(CI) 711.3 (M + 1)⁺
¹H-NMR (CDCl₃)

| δ Values [ppm] | Multiplicity | Relative Intensity | Identification |
|---|---|---|---|
| 7.30−7.01 | m | 9H | Phenyl—H |
| 4.91 | d, J=16.1 Hz | 2H | —O—CH₂—COOR |
| 4.80 | d, J=16.1 Hz | | |
| 3.92 | s | 2H | Phenyl—CH₂-Phenylene- |
| 3.83 | q, J=7.1 Hz | 1H | Phenyl—CH—CH₃ |
| 2.58 | t, J=6.6 Hz | 2H | 4-CH₂ (Chroman) |
| 2.08 | s | 3H | 5-CH₃, 7-CH₃, 8-CH₃, |
| 1.98 | s | 3H | |
| 1.94 | s | 3H | |
| 1.85−1.73 | m | 2H | 3-CH₂ (Chroman) |
| 1.59−1.02 | m | 24H | Phenyl—CH—CH₃, CH, CH₂ |
| 1.23 | s | 3H | 2-CH₃ (Chroman) |
| 0.89−0.83 | m | 12H | 4 × CH₃ |
| | | | (Tocopherol Side Chain) |

| δ Values | | Relative | |
|---|---|---|---|
| [ppm] | Multiplicity | Intensity | Identification |
| 7.30-7.01 | m | 9H | Phenyl-H |
| 4.91 | d, J = 16.1 Hz | 2H | —O—CH₂—COOR |
| 4.80 | d, J = 16.1 Hz | | |
| 3.92 | s | 2H | Phenyl-CH₂-Phenylene- |
| 3.83 | q, J = 7.1 Hz | 1H | Phenyl-CH—CH₃ |
| 2.58 | t, J = 6.6 Hz | 2H | 4-CH₂ (Chroman) |
| 2.08 | s | 3H | 5-CH₃, 7-CH₃, 8-CH₃, |
| 1.98 | s | 3H | |
| 1.94 | s | 3H | |
| 1.85-1.73 | m | 2H | 3-CH₂ (Chroman) |
| 1.59-1.02 | m | 24H | Phenyl-CH—CH₃, CH, |
| | | | CH₂ |
| 1.23 | s | 3H | 2-CH₃ (Chroman) |
| 0.89-0.83 | m | 12H | 4 × CH₃ |
| | | | (Tocopherol Side Chain) |

The chemical compounds according to the invention that contain tocopherol and at least one other pharmaceutical active ingredient are suitable for healing or prophylaxis of, in particular, inflammatory diseases because of their different pharmaceutical active ingredient groups, since the pharmaceutical active ingredient that is selected preferably from the group of non-steroidal anti-inflammatory agents reduces or even interrupts the inflammatory process, whereas the tocopherol radical acts as an antioxidant. In these chemical compounds, the pharmaceutical active ingredient that is used as well as the tocopherol that is used are linked to one another either directly or via a spacer. When used as a pharmaceutical agent or prodrug, this chemically-attached combination of two pharmaceutical active ingredients produces a higher degree of effectiveness or an increased compatibility for the patients. These advantageous effects can be used in particular in the case of long-term therapy, as is necessary, i.a., in diseases of the central nervous system.

1. Chemical compounds in the form of their racemates, enantiomers or diastereomers with general formula I 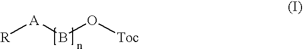
in which R stands for the unchanged portion of a variable pharmaceutical active ingredient molecule, B stands for a spacer, and Toc with the formula 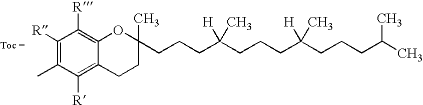
and R′, R″ and R′″ are equal to H or methyl stands for tocopherol and A stands for C═X, SOₘ, X or CH₂, whereby X is equal to 0, S or NR¹ (when n≧1) or S or NR¹ (when n=0), and B means a grouping X—R²—Y with Y equal to C═X, SOₘ or C(XR³)R⁴, and n is equal to 0 to 6, preferably 0, 1, 2 or 3, and m stands for 1 or 2, whereby R¹ stands for H, C₁ to C₁₀-alkyl, preferably C₁ to C₆-alkyl or aryl, Het or an aryl or Het radical that is bonded via a C₁ to C₆-spacer, preferably C₁ to C₃, and whereby R² is selected from the group alkylene, arylene or Het spacers as well as combinations thereof, whereby the latter are linked to one another either directly or via radical A or via grouping Xₒ-A-Xₚ, whereby o and p are equal to 0, 1 or 2, and the latter can be the same or different, and whereby R³ and R⁴ stand for H, C₁ to C₁₀-alkyl, preferably C₁-C₆-alkyl or aryl, Het or an aryl or Het radical that is bonded via a C₁ to C₆ spacer, preferably C₁ to C₃-spacers.

2. Compound of general formula I according to claim 1, in which R-A for A equal to C═O refers to an acyl radical of a pharmaceutical active ingredient from the group of the non-steroidal anti-inflammatory agents.

3. Compound according to claim 2, in which R-A for A equal to C═O represents an acyl radical of a non-steroidal anti-inflammatory agent that is selected from the group of acetylsalicylic acid, diclofenic acid, ibuprofen, indomethacin, ketoprofen, mefenamic acid, naproxen as well as derivatives thereof, especially reduction products of indomethacin as well as of ketoprofen.

4. Chemical compound of general formula I according to claim 1, in which radicals R¹, R², R³, and R⁴ are selected from the group of hydrocarbons that are unbranched, branched or cyclic, saturated or partially unsaturated with double and/or triple bond(s), unsubstituted or substituted in at least one place, preferably with F, Cl, Br, CN, NO₂, NR⁶R⁷, CHO, SOₘalkyl, OR⁶, COR⁶, COOR⁶, COCOR⁶, as well as CONR⁶R⁷.

5. Chemical compound of general formula I according to claim 1, in which the aryl radical is selected from the group of phenyl radicals that are unsubstituted or are substituted in at least one place, preferably with F, Cl, Br, CN, alkyl, CF₃, NO₂, NR⁶R⁷, CHO, SOₘalkyl, OH, OR⁶, COR⁶, COOR⁶, COCOR⁶, CONR⁶R⁷, or CSNR⁶R⁷ or are aryl- or Het-substituted, whereby the phenyl radical is optionally condensed with other cyclic compounds.

6. Chemical compounds of general formula I, whereby the alkyl or aryl radicals have the meaning according to claim 4, characterized in that radicals R⁶ and R⁷ stand for H, C₁ to C₁₀-alkyl, aryl, heteroaryl or for a C₁ to C₆-spacer-bonded aryl or heteroaryl radical.

7. Chemical compound of general formula I according to claim 2, wherein the Het radical represents a compound that is selected from the group of saturated, unsaturated or aromatic monocyclic or bicyclic heterocyclic compounds with 5 to 10 ring members as well as at least one heteroatom, preferably nitrogen, oxygen and/or sulfur, whereby optionally the heterocyclic compound is fused to another carbocyclic compound or heterocyclic compound.

8. Chemical compounds in the form of their racemates, enantiomers or diastereomers with general formula I (1) 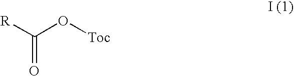
in which R and Toc have the meaning according to claim 1.

9. Chemical compounds in the form of their racemates, enantiomers or diastereomers with general formula I (2) 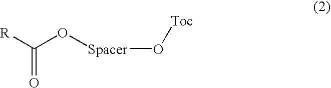
in which R, spacer, as well as Toc have the meaning according to claim 1.

10. Chemical compounds in the form of their racemates, enantiomers or diastereomers of general formula I (3) 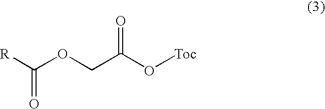
in which R and Toc have the meaning according to claim 1.

11. Carrier-linked prodrugs for non-steroidal anti-inflammatory agents and tocopherol containing at least one chemical compound according to claim 1 in isolated form or as a physiologically harmless salt and/or solvate.

12. Combined bioprecursor-carrier prodrugs containing at least one chemical compound according to claim 1 in isolated form or as a physiologically harmless salt and/or solvate.

13. Combined bioprecursor-carrier prodrugs according to claim 12, wherein one or more additional derivatizations are performed on pharmaceutically active radical R or on the tocopherol radical.

14. Process for the production of a combined bioprecursor-carrier prodrug according to claim 12, wherein the function that is characteristic of the bioprecursor is introduced by reduction reaction.

15. Pharmaceutical agent containing at least one chemical compound according to claim 1 in isolated form or as a physiologically harmless salt and/or solvate.

16. Process for the production of a pharmaceutical agent according to claim 15, wherein at least one chemical compound in isolated form and/or in the form of the corresponding physiologically harmless salt and/or solvate is mixed with additives that are common in pharmaceutics.

17. Use of a chemical compound according to claim 1 in isolated form or in the form of the corresponding physiologically harmless salts and/or solvates for the production of pharmaceutical agents for treatment or prophylaxis of degenerative diseases of the central nervous systems, such as Alzheimer's disease, Lewy body dementia, Parkinson's disease, Huntington's disease (chorea), multisystem atrophy and other similar diseases.

18. Use of a chemical compound according to claim 1 in isolated form or in the form of the corresponding physiologically harmless salts and/or solvates for the production of pharmaceutical agents for treatment of pain conditions or inflammation reactions, in particular for long-term therapy of chronic conditions.

19. Use of a chemical compound according to claim 1 in isolated form or in the form of corresponding physiologically harmless salts and/or solvates for the production of pharmaceutical agents that can be administered in a peroral, transdermal, transmucosal, rectal, inhalational, or intracerebroventricular manner.

20. Use of a chemical compound according to claim 1 in isolated form or in the form of the corresponding physiologically harmless salts and/or solvates for the production of pharmaceutical agent administration forms that are suitable for implants and/or injections and/or infusions.
